(19) United States
(12) Patent Application Publication
(10) Pub. No.: US 2024/0169208 A1
(43) Pub. Date: May 23, 2024

(54) AUTOMATIC SLEEP STAGE CLASSIFICATION SYSTEM AND METHOD FOR REDUCING PERFORMANCE VARIATION AMONG USERS USING CONTRAST LEARNING METHOD

(71) Applicant: Korea University Research and Business Foundation, Seoul (KR)

(72) Inventors: Seong Whan LEE, Seoul (KR); Heon Gyu KWAK, Namyangju-si (KR); Young Seok KWEON, Incheon (KR); Gi Hwan SHIN, Daegu (KR); Ha Na JO, Seoul (KR)

(21) Appl. No.: 18/515,872

(22) Filed: Nov. 21, 2023

(30) Foreign Application Priority Data
Nov. 21, 2022 (KR) 10-2022-0156746
Aug. 18, 2023 (KR) 10-2023-0108141

(51) Int. Cl. G06N 3/0895; G06N 3/045; G16H 50/20; G16H 50/30

(52) U.S. Cl. CPC G06N 3/0895; G06N 3/045; G16H 50/20; G16H 50/30

(57) ABSTRACT

An automatic sleep stage classification system for reducing the variation in performance between users using a contrastive learning method according to one embodiment of the present invention includes: a user terminal that measures a user's biosignal and preprocesses the user's measured biosignal; and a classification server that receives the user's preprocessed biosignal from the user terminal, extracts the user's unique biosignal feature, extracts a similar feature by comparing the user's extracted unique biosignal feature and the user's biosignal feature for contrastive learning, and classifies sleep stages based on the extracted similar feature, wherein the user's biosignal includes at least one of the user's electroencephalography (EEG), electrooculography (EOG), electrocardiogramalectromyography (EMG), respiratory effort signals, pulse, oxygen saturation (SpO2), and blood flow.

### CROSS-REFERENCE TO RELATED PATENT APPLICATION

This application claims the benefit under 35 U.S.C. 119(a) of Korean Patent Application No. 10-2022-0156746 filed on Nov. 21, 2022 and Korean Patent Application No. 10-2023-0108141 filed on Aug. 18, 2023 in the Korean Intellectual Property Office, the entire disclosure of which is incorporated herein by reference for all purposes.

### BACKGROUND OF THE INVENTION

### 1. Field of the Invention

The present invention relates to an automatic sleep stage classification system and method. More specifically, the present invention relates to an automatic sleep stage classification system and method for reducing the variation in performance between users using a contrastive learning method.

### 2. Description of the Related Art

Sleep experts evaluate the quality of sleep of patients through polysomnography and diagnose various sleep disorders. Sleep experts use polysomnography to record various physiological signals, such as patients' electroencephalography (EEG), electrooculography (EOG), electrocardiogramalectromyography (EMG), respiratory effort signals, pulse, oxygen saturation (SpO2), blood flow, etc. and based on these biosignals, classify the patent's sleep stages according to the guidelines of the American Academy of Sleep Medicine (AASM) into wakefulness (Wake), non-rapid eye movement stage 1 (Non-REM1), non-rapid eye movement stage 2 (Non-REM2), non-rapid eye movement stage 3 (Non-REM3), and rapid eye movement (REM) sleep, for every 30 seconds of sleep.

However, this passive method requires a high level of medical expertise and labor from sleep experts and also requires a significant amount of time resources.

Therefore, research on automatic sleep stage classification systems has been proposed to assist sleep experts and also enables general users to self-assess their sleep quality without the need for professional knowledge (A. Supratak et al., 2017).

Based on such research, current smart healthcare markets have commercialized smart wearable devices such as Apple Watch, Fitbit, Galaxy Watch, Mi Band, etc. that incorporate automatic sleep stage classification systems.

These systems primarily classify sleep stages using biosignal measurement technology, called photoplethysmography (PPG), and body acceleration sensors, which, however, are known to have lower accuracy than those based on electroencephalography (EEG), which is a biosignal commonly used in research on automatic classification of sleep stages (L. R. Rentz et al., 2021 and K. Kim et al., 2021).

With the advancement of deep learning technology, EEG-based automatic sleep stage classification systems demonstrate performance levels similar to human sleep experts when using specific datasets in controlled environments (J. P. Baker et al., 2022).

However, despite the high performance, EEG-based automatic sleep stage classification systems have not yet reached the level of commercialization. One of the reasons for this is the significant variation in performance when these biosignal-based systems operate based on various datasets in uncontrolled situations, such as our daily lives (A. Guillot et al., 2021).

Therefore, there is a need to develop an automatic sleep stage classification system that can reduce the variation in performance between patients so that the automatic sleep stage classification system can achieve meaningful performance and reliability that can be used in our daily lives.

Furthermore, for the purpose of automatic sleep stage classification, existing inventions have focused on different approaches to diversifying, extracting and analyzing biosignals. These approaches may exhibit high performance in specific environments or for specific users, but they have limitations in consistently delivering reliable performance across various situations and users.

In terms of domestic technologies that automatically measure sleep stages using biosignals, there are methods utilizing pulse oximetry, methods based on the user's level of consciousness using brain connectivity, methods utilizing fractal properties of heart rate variability, methods employing brainwaves, electrooculography, and electrocardiogramell as methods that extract features of biosignals through deep learning to classify sleep stages. The above-mentioned technologies utilize various biosignals and analysis methods, but there is a lack of methods for reducing the variation in performance between users of the automatic sleep stage classification system.

In terms of overseas technologies for automatic sleep stage classification, there are methods that classify sleep stages based on the user's electroencephalogram and sleep cycle patterns, methods utilizing both electroencephalogram and electrocardiogram, methods utilizing the user's pulse, methods based on the user's respiratory rate and respiratory variability, as well as methods classifying sleep stages by measuring fatigue based on electrooculography. However, these technologies also have limitations in that there is a lack of methods for reducing the variation in performance between users of the automatic sleep stage classification system.

### SUMMARY OF THE INVENTION

An object of the present invention is to provide an automatic sleep stage classification system and method for reducing the variation in performance between users using a contrastive learning method, which can solve the above-described limitations and problems, can be useful for real-life applications by addressing the problem of unstable classification performance depending on operational environments and user variations, can induce more robust and stable performance against user variations compared to existing methods by conducting classification based on biosignal features shared among multiple users, and can also reduce the variation in performance between users, which is inherent in machine learning-based automatic sleep stage classification systems.

The above-mentioned objects of the present invention are not limited to those mentioned above, and other objects not mentioned will be clearly understood by those skilled in the art from the following description.

To achieve the above-mentioned objects, one embodiment of the present invention provides an automatic sleep stage classification system for reducing the variation in performance between users using a contrastive learning method, the system comprising: a user terminal that measures a user's biosignal and preprocesses the measured user's biosignal; and a classification server that receives the user's preprocessed biosignal from the user terminal, extracts the user's unique biosignal feature, extracts a similar feature by comparing the user's extracted unique biosignal feature and the user's biosignal feature for contrastive learning, and classifies sleep stages based on the extracted similar feature, wherein the user's biosignal includes at least one of the user's electroencephalography (EEG), electrooculography (EOG), electrocardiogramalectromyography (EMG), respiratory effort signals, pulse, oxygen saturation (SpO2), and blood flow

According to one embodiment, the user terminal may comprise: a biosignal measurement unit that measures the user's biosignal; a preprocessing unit that removes noise and preprocesses the user's measured biosignal (hereinafter referred to as the user's biosignal measurement data); and a first communication unit that transmits the user's biosignal measurement data, which has been noise-removed and preprocessed by the preprocessing unit, to the classification server.

According to one embodiment, the classification server may comprise: a second communication unit that receives the user's biosignal measurement data that has been noise-removed and preprocessed (hereinafter referred to as the user's preprocessed biosignal measurement data) from the first communication unit; a memory unit that stores the user's preprocessed biosignal measurement data; and a first feature extraction unit that extracts the user's unique biosignal feature from the user's preprocessed biosignal measurement data.

According to one embodiment, the classification server may comprise a contrastive learning execution unit that performs contrastive learning on the user's biosignal measurement data for contrastive learning pre-stored in the memory unit and the user's biosignal measurement data for contrastive learning has been noise-removed and processed and includes a plurality of users' biosignal measurement data.

According to one embodiment, the classification server may comprise a second feature extraction unit that extracts the user′ unique biosignal feature for contrastive learning from the user's biosignal measurement data for contrastive learning pre-stored in the memory unit.

According to one embodiment, the classification server may comprise a similar feature extraction unit that extracts a similar feature (hereinafter referred to as a mutually invariant biosignal feature between users) by comparing the user's unique biosignal feature extracted by the first feature extraction unit and the user′ unique biosignal feature for contrastive learning extracted by the second feature extraction unit.

According to one embodiment, the classification server may comprise a sleep stage classification unit that classifies the user's sleep stages based on the user's unique biosignal feature extracted by the first feature extraction unit and the mutually invariant biosignal feature between users extracted by the similar feature extraction unit.

According to one embodiment, the user terminal may receive the user's sleep stage classification result from the classification server.

According to one embodiment, the user terminal may comprise a display unit that outputs the user's sleep stage classification result received from the classification server.

To achieve the above-mentioned objects, another embodiment of the present invention provides an automatic sleep stage classification method for reducing the variation in performance between users using a contrastive learning method, the method comprising the steps of: measuring, by a user terminal, a user's biosignal; preprocessing, by the user terminal, the user's measured biosignal; receiving, by a classification server, the user's preprocessed biosignal from the user terminal; extracting, by the classification server, the user's unique biosignal feature from the user's preprocessed biosignal; extracting, by the classification server, a similar feature by comparing the user's unique biosignal feature and the user's biosignal feature for contrastive learning; and classifying, by the classification server, sleep stages based on the extracted similar feature, wherein the user's biosignal includes at least one of the user's electroencephalography (EEG), electrooculography (EOG), electrocardiogra (ECG), electromyography (EMG), respiratory effort signals, pulse, oxygen saturation (SpO2), and blood flow.

According to the present invention as described above, the automatic sleep stage classification system and method for reducing the variation in performance between users using a contrastive learning method can solve the above-described limitations and problems, can be useful for real-life applications by addressing the problem of unstable classification performance depending on operational environments and user variations, can induce more robust and stable performance against user variations compared to existing methods by conducting classification based on biosignal features shared among multiple users, and can also reduce the variation in performance between users, which is inherent in machine learning-based automatic sleep stage classification systems.

The effects of the present invention are not limited to the effects mentioned above, and other effects not mentioned will be clearly understood by those skilled in the art from the description below.

### BRIEF DESCRIPTION OF THE DRAWINGS

The above and other feature and advantages of the present invention will become more apparent by describing in detail exemplary embodiments thereof with reference to the attached drawings in which:

FIG. 1 is a diagram illustrating the overall configuration of an automatic sleep stage classification system for reducing the variation in performance between users using a contrastive learning method according to a first embodiment of the present invention;

FIG. 2 is a diagram schematically illustrating the overall configuration of the automatic sleep stage classification system for reducing the variation in performance between users using a contrastive learning method according to the first embodiment of the present invention;

FIG. 3 is a diagram illustrating the structure of a user terminal in the automatic sleep stage classification system for reducing the variation in performance between users using a contrastive learning method according to the first embodiment of the present invention;

FIG. 4 is a diagram illustrating the structure of a classification server in the automatic sleep stage classification system for reducing the variation in performance between users using a contrastive learning method according to the first embodiment of the present invention;

FIG. 5 is a diagram illustrating the structure of a Siamese network in the automatic sleep stage classification system for reducing the variation in performance between users using a contrastive learning method according to the first embodiment of the present invention; and

FIG. 6 is a flowchart illustrating the main steps of an automatic sleep stage classification method for reducing the variation in performance between users using a contrastive learning method according to a second embodiment of the present invention.

### DETAILED DESCRIPTION OF THE INVENTION

Details regarding the objects and technical feature of the present invention and the resulting effects will be more clearly understood from the following detailed description based on the drawings attached to the specification of the present invention. Preferred embodiments according to the present invention will be described in detail with reference to the attached drawings.

The embodiments disclosed in this specification should not be construed or used as limiting the scope of the present invention. It is obvious to those skilled in the art that the description, including the embodiments, of this specification has various applications. Therefore, any embodiments described in the detailed description of the present invention are illustrative to better illustrate the present invention and are not intended to limit the scope of the present invention to the embodiments.

The functional blocks shown in the drawings and described below are only examples of possible implementations. In other implementations, different functional blocks may be used without departing from the spirit and scope of the detailed description. Moreover, although one or more functional blocks of the present invention are shown as individual blocks, one or more of the functional blocks of the present invention may be a combination of various hardware and software components that perform the same function.

Furthermore, the term “comprising” certain components, which is an “open-ended” term, simply refers to the presence of the corresponding components, and should not be understood as excluding the presence of additional components.

In addition, if a specific component is referred to as being “connected” or “coupled” to another component, it should be understood that it may be directly connected or coupled to another other component, but there may be other components therebetween.

Hereinafter, preferred embodiments of the present invention will be described in detail with reference to the accompanying drawings.

The embodiments t throughout the specification are merely exemplary embodiments to achieve the objects of the present invention, and it is understood that some components may be added or deleted as needed and one component's role may be performed in conjunction with another component.

The automatic sleep stage classification system 10 for reducing the variation in performance between users using a contrastive learning method according to the first embodiment of the present invention may comprise a processor (not shown), a network interface (not shown), a memory (not shown), a storage (not shown), and a data bus (not shown) connecting these components. Moreover, it may also include other additional components required to achieve the objects of the present invention.

The processor (not shown) may control the overall operation of each component. The processor (not shown) may be any one of a central processing unit (CPU), a microprocessor unit (MPU), a microcontroller unit (MCU), or an artificial intelligence processor commonly known in the art to which the present invention pertains. Furthermore, the processor (not shown) may perform operations for at least one application or program to perform the various functions which will be described with respect to the automatic sleep stage classification system 10 for reducing the variation in performance between users using a contrastive learning method according to the first embodiment of the present invention.

The network interface (not shown) may support wired and wireless Internet communications for the automatic sleep stage classification system 10 for reducing the variation in performance between users using a contrastive learning method according to the first embodiment of the present invention and may also support other known communication methods. Therefore, the network interface (not shown) may be configured to include a corresponding communication module.

The memory (not shown) may store various information, commands and/or information and load one or more computer programs (not shown) from the storage (not shown) to perform an automatic sleep stage classification method for reducing the variation in performance between users using a contrastive learning method according to a second embodiment of the present invention. The memory (not shown) may be composed of RAM, but it should be noted that various storage media can also be used as the memory (not shown).

The storage (not shown) may non-temporarily store one or more computer programs (not shown) and large-capacity network information (not shown). This storage (not shown) may be any one of a nonvolatile memory, such as a read only memory (ROM), an erasable programmable ROM (EPROM), an electrically erasable programmable ROM (EEPROM), and a flash memory, a hard disk drive (HDD), a solid-state drive (SSD), a removable disk, or a computer-readable recording medium commonly known in the art to which the present invention pertains.

The data bus (not shown) serves as a pathway for the movement of commands and/or information between the processor (not shown), the network interface (not shown), the memory (not shown), and the storage (not shown) as described above.

The automatic sleep stage classification system 10 for reducing the variation in performance between users using a contrastive learning method according to the first embodiment of the present invention as briefly described above may be in the form of a stand-alone device, for example, an electronic device or a server (including a cloud server). In this context, the electronic devices may include not only devices such as desktop PCs and server devices that are fixedly installed and used in one place, but also portable devices that are easy to carry, such as smartphones, tablet PCs, laptop PCs, PDAs, and PMPs, and it is suitable for any electronic device that includes a CPU corresponding to the processor (not shown) and has a network function.

Moreover, throughout the specification, the user terminal 100 may include not only devices such as desktop PCs and server devices that are fixedly installed and used in one place, but also portable devices that are easy to carry, such as smartphones, tablet PCs, laptop PCs, PDAs, and PMPs.

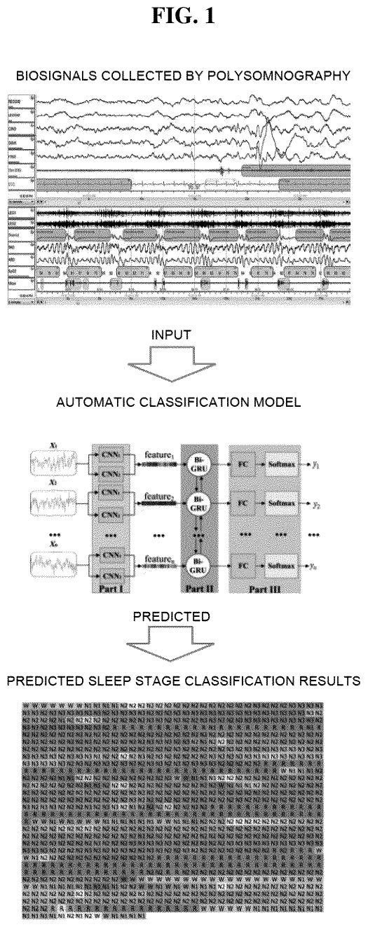

FIG. 1 is a diagram illustrating the overall configuration of an automatic sleep stage classification system for reducing the variation in performance between users using a contrastive learning method according to a first embodiment of the present invention.

Referring to FIG. 1, the automatic sleep stage classification system 10 for reducing the variation in performance between users using a contrastive learning method according to the first embodiment of the present invention can perform an automatic classification model using biosignals collected by polysomnography and can output sleep stage classification results, which are predicted as a result of performing the automatic classification model, from a user terminal 100, allowing users to verify the predicted sleep stage classification results.

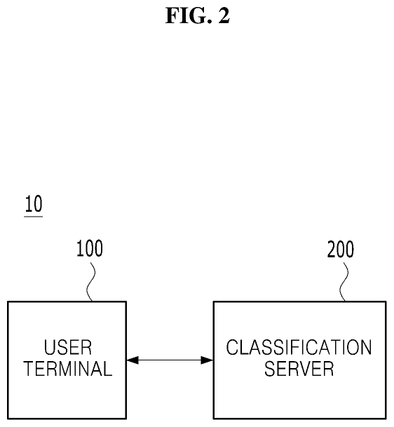

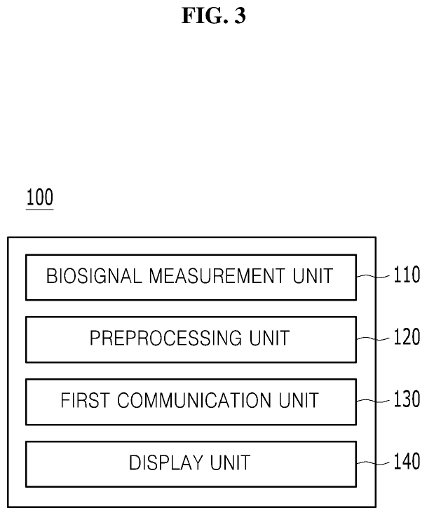

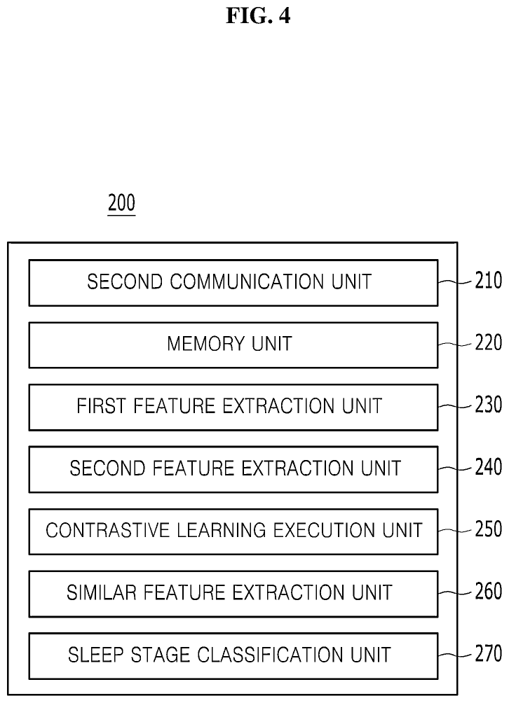

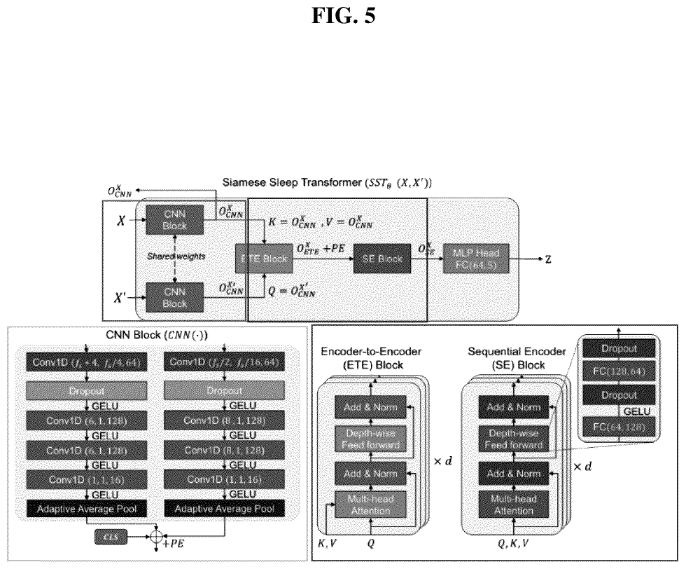

FIG. 2 is a diagram schematically illustrating the overall configuration of the automatic sleep stage classification system for reducing the variation in performance between users using a contrastive learning method according to the first embodiment of the present invention; FIG. 3 is a diagram illustrating the structure of a user terminal in the automatic sleep stage classification system for reducing the variation in performance between users using a contrastive learning method according to the first embodiment of the present invention; FIG. 4 is a diagram illustrating the structure of a classification server in the automatic sleep stage classification system for reducing the variation in performance between users using a contrastive learning method according to the first embodiment of the present invention; and FIG. 5 is a diagram illustrating the structure of a Siamese network in the automatic sleep stage classification system for reducing the variation in performance between users using a contrastive learning method according to the first embodiment of the present invention.

Referring to FIG. 2, the automatic sleep stage classification system 10 for reducing the variation in performance between users using a contrastive learning method may comprise a user terminal 100 and a classification server 200.

The data communication between the user terminal 100 and the classification server 200 can be made through wired or wireless communication. The communication method may include both wired and wireless communications, and it is not limited to any specific communication method.

The user terminal 100 may measure a user's biosignal and preprocess the user's measured biosignal.

Here, the user's biosignal may include at least one of the user's electroencephalography (EEG), electrooculography (EOG), electrocardiogra electromyography (EMG), respiratory effort signals, pulse, oxygen saturation (SpO2), and blood flow.

The classification server 200 may receive the user's preprocessed biosignal from the user terminal 100.

Moreover, the classification server 200 may extract the user's unique biosignal feature from the user's preprocessed biosignal that can be received from the user terminal 100.

Furthermore, the classification server 200 may extract a similar feature by comparing the user's extracted unique biosignal feature and the user's biosignal feature for contrastive learning.

Throughout the specification, the user's biosignal measurement data for contrastive learning has been noise-removed and processed and includes a plurality of users' biosignal measurement data.

Additionally, the classification server 200 may classify sleep stages based on the similar feature extracted by comparing the user's extracted unique biosignal feature and the user's biosignal feature for contrastive learning.

Referring to FIG. 3, the user terminal 100 may comprise a biosignal measurement unit 110, a preprocessing unit 120, a first communication unit 130, and a display unit 140.

Moreover, referring to FIG. 4, the classification server 200 may comprise a second communication unit 210, a memory unit 220, a first feature extraction unit 230, a second feature extraction unit 240, a contrastive learning execution unit 250, a similar feature extraction unit 260, and a sleep stage classification unit 270. More specifically, the biosignal measurement unit 110 can measure the user's biosignal.

The biosignal measurement unit 110 may be implemented as a wearable device that the user can wear and may include a sensor module capable of measuring the user's biosignals.

The preprocessing unit 120 may remove noise and preprocess the user's measured biosignal (hereinafter referred to as the user's biosignal measurement data).

Here, the noise removed by the preprocessing unit 120 may refer to the noise that may be generated by the user's movement, tossing and turning, etc. during the measurement of the user's biosignal.

The preprocessing performed by the preprocessing unit 120 may include signal processing such as downsampling, bandpass filtering, etc.

The first communication unit 130 may transmit the user's biosignal measurement data, which has been noise-removed and preprocessed by the preprocessing unit 120, to the classification server 200.

The second communication unit 210 may receive the user's biosignal measurement data, which has been noise-removed and preprocessed by the preprocessing unit 120 (hereinafter referred to as the user's preprocessed biosignal measurement data), from the first communication unit 130.

The memory unit 220 may the user's preprocessed biosignal measurement data.

The first feature extraction unit 230 may extract the user's unique biosignal feature from the user's preprocessed biosignal measurement data.

In more detail, the first feature extraction unit 230 may extract the user's unique biosignal feature by passing the user's preprocessed biosignal measurement data through a convolutional neural network (CNN) layer, a batch normalization layer, a linear embedding layer, and a dropout layer.

The memory unit 220 may store the user's unique biosignal feature extracted.

The contrastive learning execution unit 250 may perform contrastive learning on the user's biosignal measurement data for contrastive learning pre-stored in the memory unit 220 and the user's preprocessed biosignal measurement data.

The second feature extraction unit 240 may extract the user′ unique biosignal feature for contrastive learning from the user's biosignal measurement data for contrastive learning pre-stored in the memory unit 220.

Here, the user's biosignal measurement data for contrastive learning has been noise-removed and preprocessed and may include the user's various biosignal measurement data.

In more detail, the second feature extraction unit 240 may extract the the user′ unique biosignal feature for contrastive learning by passing the user's biosignal measurement data for contrastive learning pre-stored in the memory unit 220 through the convolutional neural network (CNN) layer.

In other words, the user's biosignal measurement data for contrastive learning passed through the convolutional neural network (CNN) layer by the second feature extraction unit 240 may be an input value for a Siamese network structure or Siamese network model and may represent the users' biosignal measurement data.

Moreover, the second feature extraction unit 240 may perform learning using a distance-based loss function to ensure that the biosignals between the users share similar features.

The first feature extraction unit 230 may extract the user's unique biosignal feature, which has the same structure as the user′ unique biosignal feature for contrastive learning, by passing the user's preprocessed biosignal measurement data through the convolutional neural network (CNN) layer.

The user's unique biosignal feature extracted by the first feature extraction unit 230 and the user′ unique biosignal feature for contrastive learning extracted by the second feature extraction unit 240 may have the same structure and may possess a Siamese network structure that shares the weights of the learning parameters.

Referring to FIG. 5, a Siamese network may refer to a network structure where two identical backbone networks with the same structure and parameter weights are placed in parallel and use different input values.

In FIG. 5, the red square box represents the Siamese network, and the backbone networks to be placed in parallel to construct the Siamese network can take the form shown in the orange square box at the bottom left of FIG. 5.

Originally, contrastive learning involves using input values x and x_aug, where x_aug is a slightly altered version with a random augmentation.

These two pairs of input values are called a positive pair, and the reason for this is that x and x_aug have slightly different shapes due to the augmentation, but fundamentally contain the same content information.

If the network is trained to maximize the similarity between the positive pair, the network can be trained to extract features that are invariant to the augmentation.

However, in the present invention, instead of using x and the augmented x_aug as input values, x (data from the original domain) and x′ (data obtained from a domain other than the original domain) are used as input values.

In this specification, the domains are set as different datasets (sleep-EDF and SHHS datasets) recorded in different environments. However, since the characteristics of brainwaves vary significantly among individuals, each user can also be considered as a domain, and thus the application of this method is likely applicable in the present invention.

When conducting contrastive learning with data x and x′ from different domains using a Siamese network structure, the network can be trained to extract feature that are invariant and shared across multiple domains.

Therefore, the present invention can extract a mutually invariant biosignal feature between users based on the user's unique biosignal feature and the user′ unique biosignal feature for contrastive learning that have the above-described Siamese network structure.

The similar feature extraction unit 260 may compare the user's unique biosignal feature extracted by the first feature extraction unit 230 and the user′ unique biosignal feature for contrastive learning extracted by the second feature extraction unit 240.

Moreover, as a result of comparison, the similar feature extraction unit 260 may extract a similar feature (i.e., the mutually invariant biosignal feature between users) between the user's unique biosignal feature extracted by the first feature extraction unit 230 and the user′ unique biosignal feature for contrastive learning extracted by the second feature extraction unit 240.

Moreover, the similar feature extraction unit 260 may determine the similarity between the user's unique biosignal feature and the user′ unique biosignal feature for contrastive learning and may optimize the user's unique biosignal feature and the user′ unique biosignal feature for contrastive learning for the extraction of the mutually invariant biosignal feature between users.

Furthermore, the similar feature extraction unit 260 may extract common biosignal features between multiple users (hereinafter referred to as users' common features) from the user's unique biosignal feature and the user′ unique biosignal feature for contrastive learning as optimized, and may adjust the weights of important features in the users' common features by performing an attention mechanism in an attention mechanism layer.

In addition, after adjusting the weights of important features in the users' common features, the similar feature extraction unit 260 may perform a normalization process through a dropout layer to prevent overfitting to a specific result.

Ultimately, the similar feature extraction unit 260 may extract the mutually invariant biosignal feature between users, which is not overfit due to the normalization process, from the user's unique biosignal feature and the user′ unique biosignal feature for contrastive learning.

Here, the similar feature extraction unit 260 may perform a loss prevention process to counteract a similarity loss that may occur during the determination of the similarity.

In this case, the similarity loss that may occur may include a cosine similarity, Euclidean distance, a Mahalanobis distance, and a triplet loss.

The sleep stage classification unit 270 may classify the user's sleep stages based on the user's unique biosignal feature extracted by the first feature extraction unit 230 and the mutually invariant biosignal feature between users extracted by the similar feature extraction unit 260.

In more detail, the sleep stage classification unit 270 may classify the user's sleep stages based on the user's unique biosignal feature and the mutually invariant biosignal feature between users as input values using a classifier based on a recurrent neural network (RNN) and a multi-layer perceptron (MLP).

Here, the classification accuracy can be further improved by learning how each sleep stage is related to the previous ones through the recurrent neural network (RNN).

Moreover, the sleep stage classification unit 270 may classify the user's sleep stages by extracting sequential features of sleep stages through a model, which utilizes both the user's unique biosignal feature and the mutually invariant biosignal feature between users by a linear combination, and through a bidirectional recurrent neural network layer.

Here, the bidirectional recurrent neural network may include long short-term memory (LSTM), gated-recurrent unit (GRU), and transformer structures.

The user terminal 100 may receive the user's sleep stage classification result from the classification server 200.

That is, the second communication unit 210 may transmit the user's sleep stage classification result to the first communication unit 130, and the first communication unit 130 may receive the user's sleep stage classification result from the second communication unit 210.

The display unit 140 may output the user's sleep stage classification result received from the classification server 200.

That is, the display unit 140 may output the user's sleep stage classification result received by the first communication unit 130 from the second communication unit 210.

Throughout the specification, the user's sleep stage classification results can be derived as at least one of wakefulness (Wake), non-rapid eye movement stage 1 (Non-REM1), non-rapid eye movement stage 2 (Non-REM2), non-rapid eye movement stage 3 (Non-REM3), and rapid eye movement (REM) sleep.

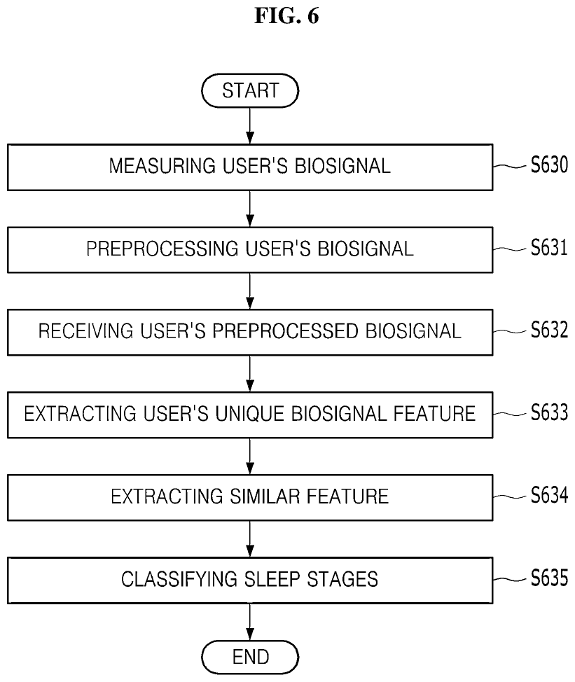

FIG. 6 is a flowchart illustrating the main steps of an automatic sleep stage classification method for reducing the variation in performance between users using a contrastive learning method according to a second embodiment of the present invention.

Referring to FIG. 6, according to the automatic sleep stage classification method for reducing the variation in performance between users using a contrastive learning method, the user terminal 100 may measure the user's biosignal (S630).

Here, the user's biosignal may include at least one of the user's electroencephalography (EEG), electrooculography (EOG), electrocardiogra electromyography (EMG), respiratory effort signals, pulse, oxygen saturation (SpO2), and blood flow.

Then, the user terminal 100 may preprocess the user's measured biosignal (S631).

Subsequently, the classification server 200 may receive the user's preprocessed biosignal from the user terminal 100 (S632).

Next, the classification server 200 may extract the user's unique biosignal feature from the user's preprocessed biosignal (S633).

Moreover, the classification server 200 may extract a similar feature by comparing the user's unique biosignal feature and the user's biosignal feature for contrastive learning (S634).

Here, the user's biosignal measurement data for contrastive learning has been noise-removed and processed and includes a plurality of users' biosignal measurement data.

Finally, the classification server 200 may classify sleep stages based on the extracted similar feature (S635).

Here, although not described in detail for the sake of avoiding redundancy, all the technical features applied to the automatic sleep stage classification system 10 for reducing the variation in performance between users using a contrastive learning method according to the first embodiment of the present invention can also be equally applied to the automatic sleep stage classification method for reducing the variation in performance between users using a contrastive learning method according to the second embodiment of the present invention.

Although the embodiments of the present invention have been described with reference to the accompanying drawings, those skilled in the art to which the present invention pertains can understand that the present disclosure can be implemented in other specific forms without changing the technical spirit or essential features thereof. Therefore, the embodiments described above should be understood as illustrative in all respects and not restrictive.

### Brief Description of Reference Numerals

10: automatic sleep stage classification system for reducing the variation in performance between users using a contrastive learning method
100: user terminal
110: biosignal measurement unit
120: preprocessing unit
130: first communication unit
140: display unit
200: classification server
210: second communication unit
220: memory unit
230: first feature extraction unit
240: second feature extraction unit
250: contrastive learning execution unit
260: similar feature extraction unit
270: sleep stage classification unit

1. An automatic sleep stage classification system for reducing the variation in performance between users using a contrastive learning method, the system comprising:
a user terminal that measures a user's biosignal and preprocesses the user's measured biosignal; and
a classification server that receives the user's preprocessed biosignal from the user terminal, extracts the user's unique biosignal feature, extracts a similar feature by comparing the user's extracted unique biosignal feature and the user's biosignal feature for contrastive learning, and classifies sleep stages based on the extracted similar feature,
wherein the user's biosignal includes at least one of the user's electroencephalography (EEG), electrooculography (EOG), electrocardiogra electromyography (EMG), respiratory effort signals, pulse, oxygen saturation (SpO2), and blood flow.

2. The automatic sleep stage classification system for reducing the variation in performance between users using a contrastive learning method of claim 1, wherein the user terminal comprises:
a biosignal measurement unit that measures the user's biosignal;
a preprocessing unit that removes noise and preprocesses the user's measured biosignal (hereinafter referred to as the user's biosignal measurement data); and
a first communication unit that transmits the user's biosignal measurement data, which has been noise-removed and preprocessed by the preprocessing unit, to the classification server.

3. The automatic sleep stage classification system for reducing the variation in performance between users using a contrastive learning method of claim 1, wherein the classification server comprises:
a second communication unit that receives the user's biosignal measurement data that has been noise-removed and preprocessed (hereinafter referred to as the user's preprocessed biosignal measurement data) from the first communication unit;
a memory unit that stores the user's preprocessed biosignal measurement data; and
a first feature extraction unit that extracts the user's unique biosignal feature from the user's preprocessed biosignal measurement data.

4. The automatic sleep stage classification system for reducing the variation in performance between users using a contrastive learning method of claim 1, wherein the classification server comprises a contrastive learning execution unit that performs contrastive learning on the user's biosignal measurement data for contrastive learning pre-stored in the memory unit and wherein the user's biosignal measurement data for contrastive learning has been noise-removed and processed and includes a plurality of users' biosignal measurement data.

5. The automatic sleep stage classification system for reducing the variation in performance between users using a contrastive learning method of claim 1, wherein the classification server comprises a second feature extraction unit that extracts the user′ unique biosignal feature for contrastive learning from the user's biosignal measurement data for contrastive learning pre-stored in the memory unit.

6. The automatic sleep stage classification system for reducing the variation in performance between users using a contrastive learning method of claim 1, wherein the classification server comprises a similar feature extraction unit that extracts a similar feature (hereinafter referred to as a mutually invariant biosignal feature between users) by comparing the user's unique biosignal feature extracted by the first feature extraction unit and the user′ unique biosignal feature for contrastive learning extracted by the second feature extraction unit.

7. The automatic sleep stage classification system for reducing the variation in performance between users using a contrastive learning method of claim 1, wherein the classification server comprises a sleep stage classification unit that classifies the user's sleep stages based on the user's unique biosignal feature extracted by the first feature extraction unit and the mutually invariant biosignal feature between users extracted by the similar feature extraction unit.

8. The automatic sleep stage classification system for reducing the variation in performance between users using a contrastive learning method of claim 1, wherein the user terminal receives the user's sleep stage classification result from the classification server.

9. The automatic sleep stage classification system for reducing the variation in performance between users using a contrastive learning method of claim 8, wherein the user terminal comprises a display unit that outputs the user's sleep stage classification result received from the classification server.

10. An automatic sleep stage classification method for reducing the variation in performance between users using a contrastive learning method, the method comprising the steps of:
measuring, by a user terminal, a user's biosignal;
preprocessing, by the user terminal, the user's measured biosignal;
receiving, by a classification server,
the user's preprocessed biosignal from the user terminal;
extracting, by the classification server, the user's unique biosignal feature from the user's preprocessed biosignal;
extracting, by the classification server, a similar feature by comparing the user's unique biosignal feature and the user's biosignal feature for contrastive learning; and
classifying, by the classification server, sleep stages based on the extracted similar feature,
wherein the user's biosignal includes at least one of the user's electroencephalography (EEG), electrooculography (EOG), electrocardiogra electromyography (EMG), respiratory effort signals, pulse, oxygen saturation (SpO2), and blood flow
